(19) United States
(12) Patent Application Publication
(10) Pub. No.: US 2024/0393321 A1
(43) Pub. Date: Nov. 28, 2024

(54) COLLOIDAL GOLD IMMUNOCHROMATOGRAPHIC JOINT INSPECTION CARD FOR DIQUAT AND PARAQUAT, PREPARATION METHOD AND APPLICATION THEREOF

(71) Applicant: The First Affiliated Hospital of Chongqing Medical and Pharmaceutical College, Chongqing (CN)

(72) Inventors: Shangcheng XU, Chongqing (CN); Guanyan FU, Chongqing (CN); Yongsheng LIU, Chongqing (CN); Yu DUAN, Chongqing (CN); Tao LI, Chongqing (CN); Wanjiang ZHAO, Chongqing (CN); Wen PEI, Chongqing (CN); Maosen ZHANG, Chongqing (CN)

(21) Appl. No.: 18/646,237

(22) Filed: Apr. 25, 2024

(30) Foreign Application Priority Data
May 25, 2023 (CN) 202310597649.5

(51) Int. Cl. G01N 33/53; G01N 30/92; G01N 30/94; G01N 33/543

(52) U.S. Cl. CPC G01N 33/5308; G01N 30/92; G01N 30/94; G01N 33/54388; G01N 2030/945

(57) ABSTRACT

The present disclosure provides a colloidal gold immunochromatographic joint inspection card for diquat and paraquat in the field of on-site rapid detection, as well as a preparation method and application. The joint inspection card includes a PVC base plate, where the PVC base plate is sequentially provided with a sample pad, a gold-labelled pad, a nitrocellulose membrane and a water-absorbing pad along the chromatographic direction; the gold-labelled pad is adsorbed with an antibody solution labeled with paraquat colloidal gold and an antibody solution labeled with diquat colloidal gold; the nitrocellulose membrane is provided with a T1 detection line, a T2 detection line and a quality control line along the chromatographic direction; and an area of the T1 detection line is coated with paraquat complete antigen, an area of the T2 detection line is coated with diquat complete antigen, and the quality control line is coated with goat anti-mouse IgG.

### CROSS-REFERENCE TO RELATED APPLICATIONS

This application claims priority to Chinese Patent Application No. 202310597649.5, filed on May 25, 2023, the contents of which are hereby incorporated by reference.

### TECHNICAL FIELD

The present disclosure belongs to the field of on-site rapid detection, and in particular to a colloidal gold immunochromatographic joint inspection card for diquat and paraquat, a preparation method and an application thereof.

### BACKGROUND

Diquat and paraquat are both bipyridine herbicides with similar composition, yet the diquat has a lower toxicity than paraquat, with a lethal dose of 6-12 grams (g) for adults and a lethality rate of 20-60%, and an inverse correlation between the damaging effect on the organism and the dosage, while the lethal dose of paraquat for adults is 5-15 milliliters (mL) of a 20% aqueous solution (20-40 milligrams per kilogram, or mg/kg) with a lethality rate of up to 90%, and a relatively low success rate of rescuing the patient. In China, the production, sale and use of paraquat has been banned since July 2016 due to the dangers of the product and the lack of an antidote or specific drug, and the application of diquat as an alternative to paraquat in agricultural production has been increasing year by year as a result of the banning of paraquat.

With lower toxicity than paraquat, diquat is less effective in weed control than paraquat and priced higher than paraquat, and some unscrupulous merchants mix paraquat with diquat or use these products under the same brand name. Paraquat and diquat possess similar structural and physicochemical properties, but their toxicity and target organs are different. Paraquat is targeted at the lungs, causing pulmonary fibrosis, seriously affecting respiratory function and is the main cause of death, while diquat mainly causes damage to the kidneys, the heart and the central nervous system of human beings, and in case of accidental ingestion of the herbicide mixture of diquat and paraquat, or paraquat counterfeited as diquat, timely testing is required to take appropriate rescue measures.

Existing methods for the detection of paraquat and diquat include gas chromatography, mass spectrometry, high performance liquid chromatography, ultraviolet spectrophotometry and capillary electrophoresis. Although the above methods are capable of quantitatively or qualitatively detecting paraquat and diquat with good specificity, these methods are not easy to be widely used in clinical practice due to the problems of complicated detection and long duration of testing, heavy reliance on testing equipment and relevant professional personnel as well as the high cost.

For example, the patent with Publication number CN111781199A discloses a method for rapid detection of paraquat and diquat in urine, including: step 1, preparing urine containing paraquat and diquat with different concentrations; step 2, preparing a color chart; step 3, preparing a test tube; step 4, preparing a reagent; step 5, combining a urine to be detected with the reagent; and step 6, observing and detecting urine in terms color change.

In this method, paraquat or diquat is detected according to the principle that paraquat and diquat contain a conjugated structure and have a pH value of 10 or more, which form free ions and produce a blue colour (paraquat) or a green colour (diquat). Since this method is cumbersome and offers a low sensitivity, and the colour differences between paraquat and diquat in urine are similar when the concentrations of paraquat and diquat are similar, causing interference with each other and affecting the results of the test, and therefore increasing the risk that the misuse of resuscitation means will affect the success rate of resuscitation, a colloidal gold immunochromatographic card for diquat and paraquat and an application thereof are proposed.

### SUMMARY

The present disclosure aims to provide a colloidal gold immunochromatographic joint inspection card for diquat and paraquat, a preparation method and an application thereof, so as to realize simultaneous rapid detection of two toxic substances, namely diquat and paraquat, and to solve the problem of the existing technology that when the concentrations of paraquat and diquat are similar, the colour development of paraquat and diquat is similar, which is easy to interfere with each other and affect the detection results.

In order to achieve the above objectives, the present disclosure adopts following technical schemes: a colloidal gold immunochromatographic joint inspection card for diquat and paraquat, including a PVC base plate, where the PVC base plate is sequentially provided with a sample pad, a gold-labelled pad, a nitrocellulose membrane and a water-absorbing pad along a chromatographic direction; the gold-labelled pad is absorbed with an antibody solution labeled with paraquat colloidal gold and an antibody solution labeled with diquat colloidal gold; the nitrocellulose membrane is provided with a T1 detection line, a T2 detection line and a quality control line along the chromatographic direction, an area of the T1 detection line is coated with paraquat complete antigen, an area of the T2 detection line is coated with diquat complete antigen, and the quality control line is coated with goat anti-mouse immunoglobulin G (IgG).

The principle and advantages of this scheme are as follows.

Rapid detection of paraquat and diquat is achieved by the application of a joint inspection card, which has the advantage of quick detection, and the simultaneous encapsulation of paraquat complete antigen and diquat complete antigen on the joint inspection card enables simultaneous detection, thus realizing the simultaneous rapid detection of paraquat and diquat.

When the sample solution containing any one of paraquat, diquat or a mixture of the two is dripped into the sample pad, and the sample solution passes through the gold-labelled pad adsorbed with antibody solution labeled with paraquat colloidal gold and antibody solution labeled with diquat colloidal gold, the paraquat component reacts with the antibody solution labeled with paraquat colloidal gold, the diquat component reacts with the antibody solution labeled with diquat colloidal gold, and the paraquat and diquat do not interact with each other when reacting with the antibody solutions labelled with colloidal gold, and are capable of binding to paraquat complete antigen and diquat complete antigen respectively when passing through the T1 detection line and the T2 detection line, so that even if the concentrations of paraquat and diquat are similar, their binding and colour development areas are different, and the detection results will not interfere with each other.

In the prior art, paraquat and diquat are similar in colour development when their concentrations are similar, and are prone to interfere with each other to affect the detection results, whereas in the present disclosure, diquat and paraquat are not interfered with each other in the detection, and the results may be accurately identified, thus avoiding false positives, which may lead to misuse of the treatment plan to affect the success rate of the emergency rescue, and thus, the present disclosure has a higher sensitivity compared with the prior art.

Moreover, there is no need to pre-treat the sample solution during the detection in this disclosure, which not only improves the detection efficiency, but also avoids the influence of mobile phase and pre-treatment solution on the detection of paraquat due to the high polarity of paraquat, as well as the double-peak of the spectrum, which affects the accuracy of the detection result. After experimental testing, the detection efficiency of the card in this disclosure is 5-10 min, the sensitivity is 20 ng/ml, the detection accuracy is 97%, the sensitivity is comparable with that of the liquid-quantity and other large-scale instruments, the error of the detection results is small, but the detection is faster than that of the liquid-quantity, providing diagnostic basis for the diagnosis of paraquat and diquat poisoning in the shortest possible time to fight for the precious time of resuscitation and increase the survival rate of the poisoned patients, and it is suitable for the rapid detection of the poisoning scene and the laboratory and the batch screening.

Further, the nitrocellulose membrane has three through-slots along its length direction, bottoms of the through-slots run through the nitrocellulose membrane and extend to the PVC base plate, a first airbag is bonded to a bottom of a side wall of the through-slots, a heat insulation layer is bonded to a middle part of the side wall of the through-slots, and side walls of the three through-slots are respectively slidingly fitted with a frame, and three frames are respectively filled with paraquat complete antigen, diquat complete antigen, and goat anti-mouse IgG, a frame filled with paraquat complete antigen constitutes the T1 detection line, a frame filled with diquat complete antigen constitutes the T2 detection line, and a frame filled with goat anti-mouse IgG constitutes the quality control line; a buffer groove is opened at a side of a top of the PVC base plate near the sample pad, a second airbag and a PAM water-absorbing membrane are bonded to the buffer groove, the first airbag is communicated with the second airbag, and in an initial state, the frames are located in a middle part of the side walls of the through-slots, and the first airbag and the second airbag are in a state of fullness.

### BENEFICIAL EFFECTS

This scheme is provided with a buffer groove on the PVC base plate, so that when the sample solution is dripped into the sample pad, a part of the sample solution is temporarily stored in the buffer groove and PAM water-absorbing membrane, which slows down the speed of the sample solution flowing to the gold-labelled pad, and avoids the sample solution flow rate from being too fast, so that the components of paraquat fail to be bound to the paraquat complete antigen, or the components of diquat fail to be bound to the diquat complete antigen, thus avoiding affecting the accuracy of detection result; also, the PAM water-absorbing membrane has excellent water-absorbing ability, and when a low concentration of sample solution temporarily exists in the buffer groove, the water in the sample solution may be adsorbed by the PAM water-absorbing membrane, so that the concentration of sample solution flowing to the gold-abelled pad rises, thereby enabling the paraquat component in the sample solution to more accurately bind with the paraquat complete antigen or the diquat component to more accurately bind with the diquat complete antigen, therefore, even if the sample solution drops into the sample solution with a low concentration of paraquat or diquat, the sample solution may still be detected after the PAM water-absorbing membrane absorbs the water and concentrates the water to detect whether or not paraquat or diquat is contained in the sample solution, so as to enable the joint inspection card of the present disclosure to have a higher degree of sensitivity.

The T1 detection line, the T2 detection line and the quality control line are buried in the through-slots before the use of the joint inspection card. The insulation layer in this scheme may be selected as a cold storage material with a phase change temperature of −20° C.-0° C., so that the paraquat complete antigen, the diquat complete antigen and the goat anti-mouse IgG are insulated under the action of the insulation layer to maintain their activity and function, and to prolong the shelf-life of the joint inspection card.

The second airbag is bonded between the buffer groove and the PAM water-absorbing membrane, which may prevent the sample solution from leaking along the buffer groove, and when the buffer groove accumulates the sample solution, the second airbag is squeezed by the gravity of the sample solution, and the gas in the second airbag flows to the first airbag, and the first airbag expands, which in turn pushes the frame to slide upwards, so that the T1 detection line, the T2 detection line, and the quality control line are exposed on the surface of the nitrocellulose membrane, which is convenient for the sample solution passing through the gold-labelled pads to bind to the paraquat complete antigen, the diquat complete antigen, and the goat anti-mouse IgG, so that the sample solution may be tested for the presence of the ingredients of paraquat or diquat; the gravity of the sample solution is used to change the flow direction of the gas in the first airbag and the second airbag so as to control the position of the frames, allowing the T1 detection line, the T2 detection line, and the quality control line to be exposed only when the joint inspection card is used, which not only prolongs the shelf life of the joint inspection card, but also avoids contamination of the T1 detection line, the T2 detection line, and the quality control line in the joint inspection card to affect the results of the test.

Optionally, a preparation method of the colloidal gold immunochromatographic joint inspection card for diquat and paraquat includes:
step a, preparation of a colloidal gold solution, including preparing the colloidal gold solution with chloroauric acid and sodium citrate as raw materials;
step b, preparation of antibody solution labeled with paraquat colloidal gold and antibody solution labeled with diquat colloidal gold, including taking potassium carbonate solution to adjust a PH value of the colloidal gold solution prepared in the step a to 8.0-8.5, respectively adding paraquat and diquat monoclonal antibody into the colloidal gold solution according to a certain amount and stirring for 1 hour (h), respectively adding bovine serum albumin dropwise and stirring for 1 h to prepare labeled colloidal gold solution with a volume concentration of 0.9-1.1%, centrifuging the labeled colloidal gold solution for 15 minutes (min) at a rotating speed of 9,500-10,500 revolutions per minute (r/min), discarding a supernatant and redissolving a precipitate with a resuspension solution to prepare the antibody solution labeled with paraquat colloidal gold and antibody solution labeled with diquat colloidal gold, respectively, and storing at 4 degrees Celsius (° C.) for later use;
step c, preparing a gold-labelled pad;
step d, coating a T1 detection line, a T2 detection line and a quality control line on a nitrocellulose membrane, namely coating paraquat complete antigen, diquat complete antigen and goat anti-mouse IgG in areas of the T1 detection line, the T2 detection line and the quality control line respectively; and
step e, assembling the colloidal gold immunochromatographic joint inspection card for diquat and paraquat.

Optionally, in the step a, before preparation, glassware is rinsed by soaking in aqua regia for standby, 99 mL of distilled water and 1 mL of chloroauric acid with a mass concentration of 1% are added to an oil bath at a constant temperature of 108° C. for preheating, and when reflux water flows into a three-necked flask, 4 mL of sodium citrate with a mass concentration of 1% is added to continue stirring and heating for 30 min until the solution turns from violet to red, and the solution is cooled and stirred to room temperature to produce a colloidal gold solution containing colloidal gold particles with a particle size of 10-30 nm.

Optionally, in the step b, the resuspension solution is a mixture of 0.02-0.1 mole per liter (mol/L) Tris-HCl, 3%-7% bovine serum protein, 0.1%-0.3% TritonX-100 and 8%-12% sucrose.

Optionally, in the step c, the antibody solution labeled with paraquat colloidal gold and antibody solution labeled with diquat colloidal gold prepared in the step b are evenly mixed in proportion, and the gold-labeled pad is soaked in a mixed solution and dried at 35-40° C. for later use.

Optionally, in the step d, a concentration of paraquat complete antigen is 0.2-1.0 mg/mL, a concentration of diquat complete antigen is 0.2-1.0 mg/mL, and a concentration of goat anti-mouse IgG is 0.5-1.5 mg/mL, and the nitrocellulose membrane after coating is dried at 35-40° C. for later use.

Optionally, in the step e, the sample pad, the gold-labelled pad, the nitrocellulose membrane after coating and the water-absorbing pad are sequentially adhered to the PVC base plate, and cut into strips to prepare the colloidal gold immunochromatographic joint inspection card for diquat and paraquat.

The preparation method provided by the scheme has the following beneficial effects.

The chloroauric acid is preheated in order to increase the reaction rate on the one hand, and to promote the solubility on the other hand, facilitating the sufficient reaction with sodium citrate in order to obtain the colloidal gold particles with uniform particle size.

Potassium carbonate is a mild regulator that is capable of adjusting a wide range of pH value, and will not produce strong corrosiveness to the colloidal gold solution, so the adjustment of pH value by using potassium carbonate solution will not affect the quality of the colloidal gold solution; as potassium carbonate may cause agglomeration of some of the colloidal gold particles, centrifugation of which will enable them to be dispersed under the centrifugal action of collision with each other, and to fully bind with monoclonal antibodies of paraquat and diquat; Tris-HCl and TritonX-100 in the resuspension solution provide a better buffering effect to maintain the pH value of the solution, which enables the monoclonal antibodies of paraquat and diquat to better maintain their activity and function; the addition of bovine serum protein prevents non-specific binding and avoids the problem of false positives due to the binding of the paraquat component with the diquat antibody or the binding of the diquat component with the paraquat antibody when the prepared joint inspection card is used for the detection.

The reason for soaking the gold-labelled pad in the mixed solution is that the paraquat and diquat components will come into full contact with the mixed solution on the gold-labelled pad to avoid false positives when the prepared joint inspection card is used for the test.

Optionally, the colloidal gold immunochromatographic joint inspection card for diquat and paraquat may be applied for testing any of specimen types in PBS solution, tap water, river water, urine, whole blood, plasma and perfusate.

Beneficial effects: the colloidal gold immunochromatographic joint inspection card for diquat and paraquat proposed in this scheme is capable of meeting the rapid detection of diquat and paraquat under various sample types.

Optionally, an application method of the colloidal gold immunochromatographic joint inspection card for diquat and paraquat, including:
S1, dropping 70-100 microliters (uL) of sampling solution on the colloidal gold immunochromatographic joint inspection card for diquat and paraquat, timing and reading a result;
S2, determining the result, where a color development degree of T2 detection line of T1 detection line is inversely related to concentrations of paraquat and diquat:
(1) the T1 detection line, T2 detection line and quality control line are all colored: paraquat negative, diquat negative;
(2) the T1 detection line is not colored, while the T2 detection line and quality control line are colored: paraquat is positive and diquat is negative;
(3) the T2 detection line is not colored, but the T1 detection line and quality control line are colored: paraquat is negative and diquat is positive;
(4) the T1 detection line and T2 detection line are not colored, while the quality control line is colored: paraquat is positive and diquat is positive; and
(5) the quality control line is not colored: the joint inspection card is invalid and re-testing is required.

The method has the beneficial effects that the existence and concentration of paraquat and diquat are determined more intuitively by observing the different color development degrees of the T1 detection line, the T2 detection line and the quality control line, and reliable basis is provided for the poisoning diagnosis and rescue from paraquat and diquat poisoning; moreover, the joint detection card proposed in this solution requires no pre-treatment of the sample solution during detection, which avoids that when liquid phase detection is used, due to the high polarity of diquat, the mobile phase as well as the pre-treated solution have a great influence on the detection of diquat, and the double peaks may easily appear in the spectrum, which may affect the accuracy of the detection results.

### BRIEF DESCRIPTION OF THE DRAWINGS

FIG. 1 is an axonometric view of colloidal gold immunochromatographic joint inspection card for diquat and paraquat according to Embodiment 1 of the present disclosure, in which the arrow direction is the chromatographic direction.

FIG. 2 shows a cross-sectional view of colloidal gold immunochromatographic joint inspection card for diquat and paraquat according to Embodiment 2 of the present disclosure.

FIG. 3 is a schematic diagram for judging the results of colloidal gold immunochromatographic joint inspection card for diquat and paraquat.

FIG. 4 is a schematic diagram of the results of the colloidal gold immunochromatographic joint inspection card for diquat and paraquat when different contents of paraquat and diquat are added to blood and urine.

FIG. 5 illustrates a process of a preparation method of a colloidal gold immunochromatographic joint inspection card for diquat and paraquat.

FIG. 6 illustrates an application method of the colloidal gold immunochromatographic joint inspection card for diquat and paraquat.

### DETAILED DESCRIPTION OF THE EMBODIMENTS

A further detailed description is given below by way of specific embodiments.

The reference numerals in the attached drawings of the specification include: PVC base plate 1, sample pad 2, gold-labelled pad 3, nitrocellulose membrane 4, water-absorbing pad 5, T1 detection line 6, T2 detection line 7, quality control line 8, buffer groove 9, PAM water-absorbing membrane 10, second airbag 11, through-slot 12 and first airbag 13

### Embodiment 1

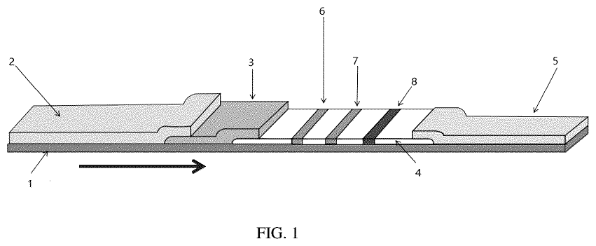

The embodiment is basically as shown in FIG. 1.

The colloidal gold immunochromatographic joint inspection card for diquat and paraquat includes a PVC base plate 1, where the PVC base plate 1 is sequentially provided with a sample pad 2, a gold-labelled pad 3, a nitrocellulose membrane 4 and a water-absorbing pad 5 along the chromatographic direction, the gold-labelled pad 3 absorbs an antibody solution labeled with paraquat colloidal gold and an antibody solution labeled with diquat colloidal gold, the nitrocellulose membrane 4 is provided with a T1 detection line 6, a T2 detection line 7 and a quality control line 8 along the chromatographic direction, the area of the T1 detection line 6 is coated with paraquat complete antigen, and the area of the T2 detection line 7 is coated with diquat complete antigen, and the quality control line 8 is coated with goat anti-mouse IgG.

### Embodiment 2

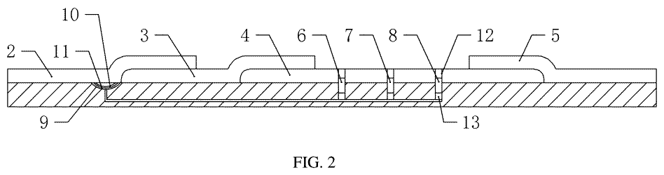

The difference from Embodiment 1 is that, as shown in FIG. 2, the nitrocellulose membrane 4 is provided with three through-slots 12 along its length direction, the bottoms of the through-slots 12 penetrate through the nitrocellulose membrane 4 and extend into the PVC base plate 1, the first airbag 13 is bonded below the side walls of the through-slots 12, and the thermal insulation layer is bonded in the middle of the side walls of the through-slots 12, and the side walls of the through-slots 12 are respectively fitted with a frame in a sliding way, and the three frames are filled with paraquat complete antigen, diquat complete antigen and goat anti-mouse IgG respectively. The frame filled with paraquat complete antigen constitutes the T1 detection line 6, the frame filled with diquat complete antigen constitutes the T2 detection line 7, and the frame filled with goat anti-mouse IgG constitutes the quality control line 8. The buffer groove 9 is opened at the top of the PVC base plate 1 on the side near the sample pad 2, and the second airbag 11 and the PAM water-absorbing membrane 10 are bonded to the buffer groove 9. The first airbag 13 communicates with the second airbag 11. In the initial state, the frame is located in the middle of the side wall of the through-slots 12, and the first airbag 13 is located in the middle of the through-slots 12.

### Embodiment 3

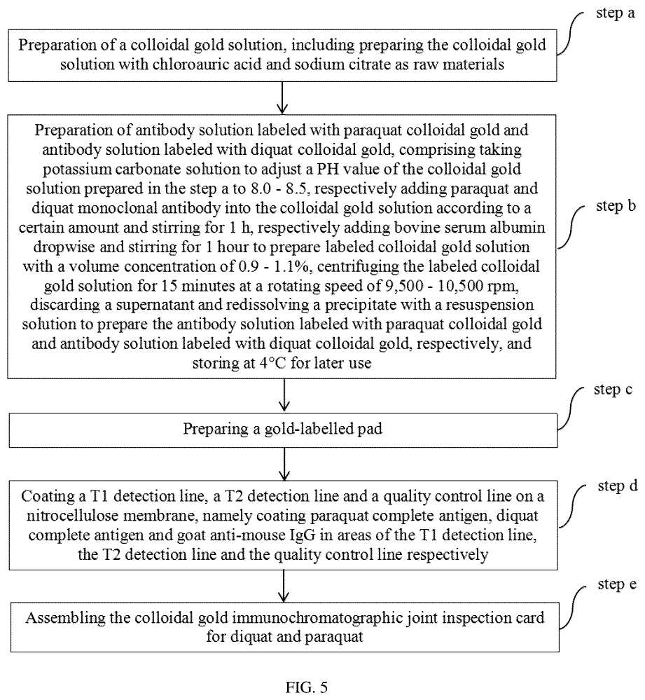

The difference from Embodiment 1 is that the preparation method of colloidal gold immunochromatographic joint inspection card for diquat and paraquat is also proposed, including following steps as shown in FIG. 5:
step a: preparation of colloidal gold solution: before preparation, glassware is rinsed by soaking in aqua regia for standby, 99 mL of distilled water and 1 mL of chloroauric acid with a mass concentration of 1% are added to an oil bath at a constant temperature of 108° C. for preheating, and when reflux water flows into a three-necked flask, 4 mL of sodium citrate with a mass concentration of 1% is added to continue stirring and heating for 30 min until the solution turns from violet to red, and the solution is cooled and stirred to room temperature to produce a colloidal gold solution containing colloidal gold particles with a particle size of 20 nm.
step b, preparation of antibody solution labeled with paraquat colloidal gold and antibody solution labeled with diquat colloidal gold, including taking potassium carbonate solution to adjust a PH value of the colloidal gold solution prepared in the step a to 8.3, respectively adding paraquat and diquat monoclonal antibody into the colloidal gold solution according to a certain amount and stirring for 1 h, respectively adding bovine serum albumin dropwise and stirring for 1 hour (h) to prepare labeled colloidal gold solution with a volume concentration of 1.0%, centrifuging the labeled colloidal gold solution for 15 minutes (min) at a rotating speed of 10,000 revolutions per minute (r/min), discarding a supernatant and redissolving a precipitate with a resuspension solution to prepare the antibody solution labeled with paraquat colloidal gold and antibody solution labeled with diquat colloidal gold, respectively, and storing at 4 degrees Celsius (° C.) for later use;

where the resuspension solution is a mixture of 0.02-0.1 mole per liter (mol/L) Tris-HCl, 5% bovine serum protein, 0.2% TritonX-100 and 10% sucrose;
step c, preparing the gold-labelled pad 3, where the antibody solution labeled with paraquat colloidal gold and antibody solution labeled with diquat colloidal gold prepared in the step b are evenly mixed in a proportion of 2:3, and the gold-labeled pad 3 is soaked in the mixed solution and dried at 37° C. for later use;
step d, coating the T1 detection line 6, the T2 detection line 7 and the quality control line 8 on the nitrocellulose membrane 4, namely coating paraquat complete antigen, diquat complete antigen and goat anti-mouse IgG in areas of the T1 detection line 6, the T2 detection line 7 and the quality control line 8 respectively;

where a concentration of the paraquat complete antigen is 0.4 mg/mL, a concentration of the diquat complete antigen is 0.6 mg/mL, and a concentration of goat anti-mouse IgG is 1.0 mg/mL, and the nitrocellulose membrane 4 after coating is dried at 37° C. for later use.

step e, sequentially adhering the sample pad 2, the gold-labelled pad 3, the nitrocellulose membrane 4 after coating and the water-absorbing pad 5 to the PVC base plate 1, and cutting into strips to prepare the colloidal gold immunochromatographic joint inspection card for diquat and paraquat.

### Embodiment 1

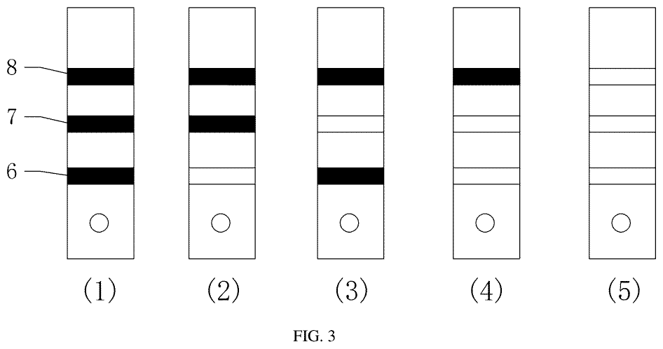

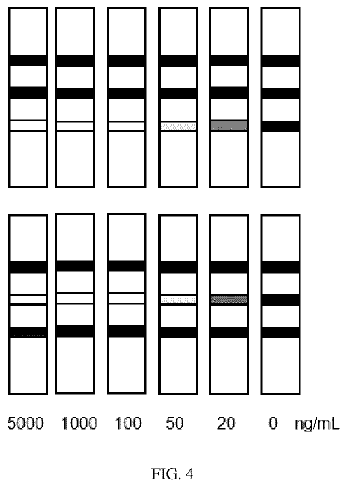

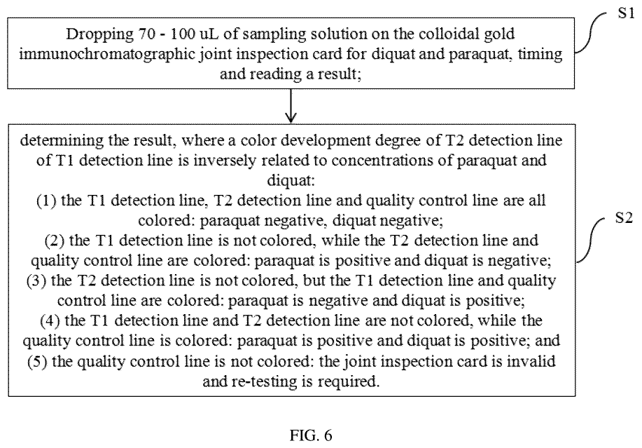

As shown in FIG. 3-FIG. 4 of the accompanying drawings, the difference with Embodiment 1 is that the present embodiment also proposes an application of the colloidal gold immunochromatographic joint inspection card for diquat and paraquat, where the colloidal gold immunochromatographic joint inspection card for diquat and paraquat may be applied for testing any of specimen types in PBS solution, tap water, river water, urine, whole blood, plasma and perfusate, with an application method as shown in FIG. 6:
S1, dropping 70-100 uL of sampling solution on the colloidal gold immunochromatographic joint inspection card for diquat and paraquat, timing and reading a result;
S2, determining the result, where a color development degree of T2 detection line 7 of T1 detection line 6 is inversely related to concentrations of paraquat and diquat:
(1) the T1 detection line 6, T2 detection line 7 and quality control line 8 are all colored: paraquat negative, diquat negative;
(2) the T1 detection line 6 is not colored, while the T2 detection line 7 and quality control line 8 are colored: paraquat is positive and diquat is negative;
(3) the T2 detection line 7 is not colored, but the T1 detection line 6 and quality control line 8 are colored: paraquat is negative and diquat is positive;
(4) the T1 detection line 6 and T2 detection line 7 are not colored, while the quality control line 8 is colored: paraquat is positive and diquat is positive; and
(5) the quality control line 8 is not colored: the joint inspection card is invalid and re-testing is required.

### The Experimental Data are as Follows

1. Sample preparation: 4000 mL of urine is taken and divided into 4 equal shares, one share is prepared by adding 20 ug paraquat to prepare a sample solution 1 with a concentration of 20 ng/mL, one share is prepared by adding 20 ug diquat to prepare a sample solution 2 with a concentration of 20 ng/mL, one share is prepared by adding 20 ug of a mixture of paraquat and diquat to prepare a sample solution 3 with a concentration of 20 ng/mL, and one share is used without adding paraquat and diquat as sample solution 4.
2. The control group and the experimental group are designed, and the sample solution is dripped respectively.

Four test tubes and four copies of diquat and paraquat colloidal gold immunochromatographic joint inspection card prepared in this scheme are taken; sample solution 1, sample solution 2, sample solution 3 and sample solution 4 are dropped into the test tubes respectively, and then placed into the liquid phase detection apparatus for detection after pre-treatment, which are used as the control group 1, control group 2, control group 3 and control group 4, while sample solution 1, sample solution 2, sample solution 3 and sample solution 4 are dropwise added to the colloidal gold immunochromatographic joint inspection card for diquat and paraquat respectively, and then allowed to stand, which are used as the experimental group 1, experimental group 2, experimental group 3 and experimental group 4.
3. Results reading and recording

The results are recorded including positive (negative) results and time of issuance of results for both control and experimental groups as shown in Table 1:

TABLE 1
Comparison of the results of sample solution 1, sample solution
2, sample solution 3 and sample solution 4 under liquid
phase detection and joint inspection card detection

| | | time of | |
|---|---|---|---|
| | | issuance | |
| Sample solution | Positive (negative) | of results | |
| Experimental group 1 | Paraquat positive, diquat negative | 7 | min |
| Experimental group 2 | Paraquat negative, diquat positive | 8 | min |
| Experimental group 3 | Paraquat positive, diquat positive | 10 | min |
| Experimental group 4 | Paraquat negative, diquat negative | 5 | min |
| Control group 1 | Paraquat positive, diquat negative | 5 | h |

| | | |
|---|---|---|
| Control group 2 | Paraquat negative, diquat positive | 5 h 15 min |
| Control group 3 | Paraquat positive, diquat positive | 5 h 38 min |
| Control group 4 | Paraquat negative, diquat negative | 4 h 20 min |

According to Table 1, the results of the experimental group and the control group correspond to the known additives of the sample solution.

In this experiment, the detection accuracy of paraquat and diquat is 100%, and the results of the experimental group are issued faster than that of the control group.

The scheme also provides a comparison table of the results of different concentrations of sample solutions under the liquid detection instruments and colloidal gold immunochromatographic joint inspection card for diquat and paraquat, as shown in Table 2:

TABLE 2
Comparison of the results of different concentrations
of sample solutions under the liquid detection instruments
and colloidal gold immunochromatographic joint inspection
card for diquat and paraquat

| | |
|---|---|
| LC-MS-MS | Joint inspection card |

| | | | |
|---|---|---|---|
| S/N of | Paraquat | Diquat | for diquat and paraquat |

| methods | (ng/mL) | (ng/mL) | Paraquat | Diquat |
|---|---|---|---|---|
| 1 | <20.0 | 20.9 | − | + |
| 2 | 203.1 | <20.0 | + | − |
| 3 | 480.6 | <20.0 | + | − |
| 4 | 654 | <20.0 | + | − |
| 5 | 44.9 | <20.0 | + | − |
| 6 | <20.0 | <20.0 | − | − |
| 7 | <20.0 | 878.2 | − | + |
| 8 | <20.0 | <20.0 | − | − |
| 9 | <20.0 | 34.0 | − | + |
| 10 | <20.0 | 345.3 | − | + |
| 11 | 6460.8 | <20.0 | + | + |
| 12 | <20.0 | 48.1 | − | + |
| 13 | 243.9 | <20.0 | + | − |
| 14 | 766.3 | <20.0 | + | − |
| 15 | 1095.4 | <20.0 | + | − |
| 16 | 307.5 | <20.0 | + | − |
| 17 | 280.0 | <20.0 | + | − |
| 18 | <20.0 | <20.0 | − | − |
| 19 | 224.6 | <20.0 | + | − |
| 20 | 202.6 | <20.0 | + | − |
| 21 | 291.2 | <20.0 | + | − |
| 22 | <20.0 | <20.0 | − | − |
| 23 | 145.4 | <20.0 | + | − |
| 24 | <20.0 | 136.7 | − | + |
| 25 | 79.9 | <20.0 | + | − |
| 26 | <20.0 | 1081.6 | − | + |
| 27 | <20.0 | <20.0 | − | − |
| 28 | 79.9 | <20.0 | + | − |
| 29 | 99.7 | <20.0 | + | − |
| 30 | 86.2 | <20.0 | + | − |
| 31 | <20.0 | 32350.7 | + | + |
| 32 | 35.3 | <20.0 | + | − |
| 33 | 28.8 | <20.0 | + | − |
| 34 | 20.4 | <20.0 | + | − |
| 35 | 21.1 | <20.0 | + | − |
| 36 | <20.0 | <20.0 | − | − |

| | | |
|---|---|---|
| Total | 97.2% | 97.2% |

Remarks: “−” means the test strip result is negative; “+” means the test strip result is positive.

The verification results show that: 1. high detection efficiency of the kit, the reaction is only 5-10 min, much lower than the 4-6 h of the traditional detection equipment; 2. high sensitivity, the lowest detection concentration is 20 ng/mL, which is comparable with the detection limit of the large laboratory equipment liquid mass spectrometer; 3. strong specificity, no cross-reaction when paraquat and diquat are detected at the same time; 4. easy operation, requiring no professional personnel and large-scale equipment, suitable for rapid detection and batch screening at the site of poisoning and in the laboratory; 5. free from matrix interference, applicable to urine, plasma, whole blood and other multi-specimen types of testing.

The research and development and application of this joint inspection card significantly enhance the rapid handling capacity of paraquat and diquat poisoning emergencies in China, and provide clinically practical rapid testing products for the clinical treatment of paraquat and diquat poisoning patients, emergency treatment of poisoning and screening of health risks.

It should be noted that in this specification, relational terms such as the first and second are only used to distinguish one entity or operation from another entity or operation, and do not necessarily require or imply that there is any such actual relationship or order between these entities or operations. Moreover, the terms “comprising”, “including” or any other variation thereof are intended to cover non-exclusive inclusion, so that a process, method, article or equipment including a series of elements includes not only those elements, but also other elements not explicitly listed or elements inherent to such process, method, article or equipment.

What have been described above are only the embodiments of the present disclosure, and the common sense of the specific structure and characteristics known in the scheme is not described here too much. Ordinary technicians in the field know all the general technical knowledge of the technical field to which the present disclosure belongs before the application date or priority date, can know all the existing technologies in the field, and have the ability to apply the conventional experimental means before the date. Under the inspiration given by this application, ordinary technicians in the field may improve and implement the scheme in combination with their own abilities. Some typical well-known structures or methods should not become ordinary in the field. It should be pointed out that for those skilled in the art, several modifications and improvements can be made without departing from the structure of the present disclosure, which should also be regarded as the protection scope of the present disclosure, and these will not affect the implementation effect of the present disclosure and the practicability of the patent. The scope of protection required by this application shall be subject to the contents of the claims, and the specific implementation in the specification can be used to explain the contents of the claims.

What is claimed is:

1. A colloidal gold immunochromatographic joint inspection card for diquat and paraquat, comprising a PVC base plate, wherein the PVC base plate is sequentially provided with a sample pad, a gold-labelled pad, a nitrocellulose membrane and a water-absorbing pad along a chromatographic direction; the gold-labelled pad is absorbed with an antibody solution labeled with paraquat colloidal gold and an antibody solution labeled with diquat colloidal gold; the nitrocellulose membrane is provided with a T1 detection line, a T2 detection line and a quality control line along the chromatographic direction, an area of the T1 detection line is coated with paraquat complete antigen, an area of the T2 detection line area is coated with diquat complete antigen, and the quality control line is coated with goat anti-mouse IgG.

2. The colloidal gold immunochromatographic joint inspection card for diquat and paraquat according to claim 1, wherein the nitrocellulose membrane has three through-slots along a length direction, bottoms of the through-slots run through the nitrocellulose membrane and extend to the PVC base plate, a first airbag is bonded to a bottom of a side wall of the through-slots, a heat insulation layer is bonded to a middle part of the side wall of the through-slots, and side walls of the three through-slots are respectively slidingly fitted with a frame, and three frames are respectively filled with paraquat complete antigen, diquat complete antigen, and goat anti-mouse IgG, a frame filled with paraquat complete antigen constitutes the T1 detection line, a frame filled with diquat complete antigen constitutes the T2 detection line, and a frame filled with goat anti-mouse IgG constitutes the quality control line; a buffer groove is opened at a side of a top of the PVC base plate near the sample pad, a second airbag and a PAM water-absorbing membrane are bonded to the buffer groove, the first airbag is communicated with the second airbag, and in an initial state, the frames are located in a middle part of the side walls of the through-slots, and the first airbag and the second airbag are in a state of fullness.

3. A preparation method of a colloidal gold immunochromatographic joint inspection card for diquat and paraquat, comprising following steps:
step a, preparation of a colloidal gold solution, comprising preparing the colloidal gold solution with chloroauric acid and sodium citrate as raw materials;
step b, preparation of antibody solution labeled with paraquat colloidal gold and antibody solution labeled with diquat colloidal gold, comprising taking potassium carbonate solution to adjust a PH value of the colloidal gold solution prepared in the step a to 8.0-8.5, respectively adding paraquat and diquat monoclonal antibody into the colloidal gold solution according to a certain amount and stirring for 1 h, respectively adding bovine serum albumin dropwise and stirring for 1 hour to prepare labeled colloidal gold solution with a volume concentration of 0.9-1.1%, centrifuging the labeled colloidal gold solution for 15 min at a rotating speed of 9,500-10,500 rpm, discarding a supernatant and redissolving a precipitate with a resuspension solution to prepare the antibody solution labeled with paraquat colloidal gold and antibody solution labeled with diquat colloidal gold, respectively, and storing at 4° C. for later use;
step c, preparing a gold-labelled pad;
step d, coating a T1 detection line, a T2 detection line and a quality control line on a nitrocellulose membrane, namely coating paraquat complete antigen, diquat complete antigen and goat anti-mouse IgG in areas of the T1 detection line, the T2 detection line and the quality control line respectively; and
step e, assembling the colloidal gold immunochromatographic joint inspection card for diquat and paraquat.

4. The preparation method of the colloidal gold immunochromatographic joint inspection card for diquat and paraquat according to claim 3, wherein in the step a, before preparation, glassware is rinsed by soaking in aqua regia for standby, 99 mL of distilled water and 1 mL of chloroauric acid with a mass concentration of 1% are added to an oil bath at a constant temperature of 108° C. for preheating, and when reflux water flows into a three-necked flask, 4 mL of sodium citrate with a mass concentration of 1% is added to continue stirring and heating for 30 min until the solution turns from violet to red, and the solution is cooled and stirred to a room temperature to produce a colloidal gold solution containing colloidal gold particles with a particle size of 10-30 nm.

5. The preparation method of the colloidal gold immunochromatographic joint inspection card for diquat and paraquat according to claim 3, wherein in the step b, the resuspension solution is a mixture of 0.02-0.1 mole per liter Tris-HCl, 3%-7% bovine serum protein, 0.1%-0.3% TritonX-100 and 8%-12% sucrose.

6. The preparation method of the colloidal gold immunochromatographic joint inspection card for diquat and paraquat according to claim 3, wherein in the step c, the antibody solution labeled with paraquat colloidal gold and antibody solution labeled with diquat colloidal gold prepared in the step b are evenly mixed in proportion, and the gold-labelled pad is soaked in a mixed solution and dried at 35-40° C. for later use.

7. The preparation method of the colloidal gold immunochromatographic joint inspection card for diquat and paraquat according to claim 3, wherein in the step d, a concentration of the paraquat complete antigen is 0.2-1.0 mg/mL, a concentration of the diquat complete antigen is 0.2-1.0 mg/mL, and a concentration of the goat anti-mouse IgG is 0.5-1.5 mg/mL, and the nitrocellulose membrane after coating is dried at 35-40° C. for later use.

8. The preparation method of the colloidal gold immunochromatographic joint inspection card for diquat and paraquat according to claim 3, wherein in the step e, the sample pad, the gold-labelled pad, the nitrocellulose membrane after coating and the water-absorbing pad are sequentially adhered to the PVC base plate, and cut into strips to prepare the colloidal gold immunochromatographic joint inspection card for diquat and paraquat.

9. An application of a colloidal gold immunochromatographic joint inspection card for diquat and paraquat, wherein the colloidal gold immunochromatographic joint inspection card for diquat and paraquat according to claim 1 is applied for testing any of specimen types in PBS solution, tap water, river water, urine, whole blood, plasma and perfusate.

10. The application of the colloidal gold immunochromatographic joint inspection card for diquat and paraquat according to claim 9, wherein an application method of colloidal gold immunochromatographic joint inspection card for diquat and paraquat is as follows:
S1, dropping 70-100 uL of sampling solution on the colloidal gold immunochromatographic joint inspection card for diquat and paraquat, timing and reading a result;
S2, determining the result, where a color development degree of T2 detection line of T1 detection line is inversely related to concentrations of paraquat and diquat:
(1) the T1 detection line, T2 detection line and quality control line are all colored: paraquat is negative, diquat is negative;
(2) the T1 detection line is not colored, while the T2 detection line and quality control line are colored: paraquat is positive and diquat is negative;
(3) the T2 detection line is not colored, but the T1 detection line and quality control line are colored: paraquat is negative and diquat is positive;
(4) the T1 detection line and T2 detection line are not colored, while the quality control line is colored: paraquat is positive and diquat is positive; and
(5) the quality control line is not colored: the joint inspection card is invalid and re-testing is required.
